(19) United States
(12) Patent Application Publication
(10) Pub. No.: US 2005/0153288 A1
(43) Pub. Date: Jul. 14, 2005

(54) Methods for identifying gibberellin responsive genes using cultured vegetable cells

(76) Inventors: Eiichi Minami, Ibaraki (JP); Naoto Shibuya, Kanagawa (JP); Robert B. Day, Berkeley, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/496,530

(22) PCT Filed: Dec. 20, 2002
(86) PCT No.: PCT/JP02/13376

(30) Foreign Application Priority Data
Dec. 21, 2001 (JP) 2001-388993

(51) Int. Cl. C12Q 1/68

(52) U.S. Cl. 435/6

(57) ABSTRACT

To date, those skilled in the art have never considered that cultured plant cells respond to gibberellins. However, to comprehensively and conveniently identify and isolate gibberellin responsive genes, cultured cells, which are uniform and easy to handle, are appropriate. Considering the advantages of cultured cells, the present inventors developed methods for identifying and isolating gibberellin responsive genes using cultured cells. After pre-culturing cells in an auxin-free medium, they examined whether or not changes in gene expression could be detected. As a result, it was surprisingly revealed that changes in the expression of some genes could be detected after gibberellin treatment.

### TECHNICAL FIELD

The present invention relates to methods for identifying and screening gibberellin responsive genes using cultured plant cells, and methods for using cultured plant cells to determine the presence or absence of gibberellin responsiveness in tested genes.

### BACKGROUND ART

Gibberellins are a group of plant hormones that play important roles in the life cycle of plants, including roles in germination, elongation, and flower bud initiation. Therefore, gibberellin responsive genes are expected to have widespread applications in the creation of transgenic crops in which the above-mentioned germination, elongation, flower bud initiation, and so on has been modified.

To date, the following have been reported as genes whose expression is induced by gibberellins: starch hydrolyzing enzyme (α-amylase) gene in germinating seeds, barley Gamyb (transcription regulator of α-amylase gene), rice OsCa-atpase (Ca²⁺-ATPase), rice UBC (Ubiquitin-conjugating enzyme), oat Aspk10 (ribosomal protein kinase), rice Os-Exp2and4 (expansin, cell wall protein), rice cycOs1,2 (mitotic cyclin), rice cdc2Os-2 (cdc2 homologue), rice R2 (cdc2 activating kinase), rice RPA1 (replication protein A1), cucumber CHRC (carotenoid associated protein), Arabidopsis GASA1 and GASA4, and cucumber CRG16 (Shinjiro Yamaguchi, “Signal transduction of plant hormones”, Plant Cell Technology Series 10, edited by Hiroo Fukuda, Yasunori Machida, Yuji Kamiya, and Tsukaho Hattori (1998) pp. 97-106). Four of the rice expansin genes, OsEXPB3, OsEXPB4, OsEXPB6, and OsEXPB11, have also been reported to be induced by gibberellins (Lee, Y., and Kende, H., Plant Physiol. (2001) 127: 645-654). In addition, expression of the following genes has been reported to be suppressed by gibberellins: genes involved in gibberellin biosynthesis, oat Aspk9 (MAP kinase), Vigna AB-ACO1 (ACC oxidase), Arabidopsis GA4 (gibberellin 3β-hydroxylase), and Arabidopsis At2301 (GA5, gibberellin 20-oxidase) (Shinjiro Yamaguchi, “Signal transduction of plant hormones”, Plant Cell Technology Series 10, edited by Hiroo Fukuda, Yasunori Machida, Yuji Kamiya, and Tsukaho Hattori (1998) pp. 97-106).

Gibberellin responsiveness has been identified by using whole plants or slices thereof. However, whole plants and slices thereof are composed of heterogeneous cells and are difficult to handle. Therefore, comprehensive, convenient identification and isolation of gibberellin responsive genes is difficult when whole plants or their slices are used.

### DISCLOSURE OF THE INVENTION

The present invention has been made under the above circumstances. An objective of this invention is to provide methods for comprehensive and convenient identification and isolation of gibberellin responsive genes.

To date, those skilled in the art had never considered that cultured plant cells can respond to gibberellins. However, cultured cells, which are uniform and easy to handle, are suited for the purpose of comprehensive and convenient identification and isolation of gibberellin responsive genes. Considering the advantages of cultured cells, the present inventors developed methods for identifying and isolating gibberellin responsive genes using cultured cells. Specifically, the inventors first tested whether any change in gene expression was detected on treating suspension-cultured cells of rice with a gibberellin, GA3. No change in gene expression was detected. To maintain cell division activity, media for plant cell culture contains auxins that are not easily metabolized in plants. Therefore, considering the possibility that auxins might suppress gibberellin's action, the present inventors examined whether any change in gene expression was detected after cells were pre-cultured in a medium lacking auxins. In a surprising result, changes in the gene expression of several genes were detected shortly after gibberellin treatment. “Gibberellin” is a general term for compounds that comprise an ent-gibberellane backbone. Only biologically active gibberellins were effective in the induction of expression of these genes. This strongly suggested that the induction of expression of these genes was mediated by signal transduction through a gibberellin receptor. Results of analysis using inhibitors of protein kinase and protein phosphatase suggested the involvement of protein kinase and protein phosphatase in the process of signal transduction leading to gene expression. In addition, a correlation was recognized between gibberellin-dependent gene expression in cultured cells and in the green leaves of whole plants. These results show that gibberellin responsive genes in whole plants can be identified and isolated using cultured cells.

In other words, for the first time in the world, the present inventors have succeeded in developing methods for identifying and isolating gibberellin responsive genes using cultured plant cells, thereby accomplishing the present invention. More specifically, the present invention provides:
[1] a method for identifying gibberellin responsive genes, wherein the method comprises the steps of:
   (a) contacting cultured plant cells with gibberellin,
   (b) detecting gene expression in the cultured plant cells contacted with gibberellin, and
   (c) identifying genes that have changes in expression when compared with cells that were not contacted with gibberellin;
[2] a method of screening for gibberellin responsive genes, wherein the method comprises the steps of:
   (a) contacting cultured plant cells with gibberellin,
   (b) detecting gene expression in the cultured plant cells contacted with gibberellin,
   (c) identifying genes that have changes in expression when compared with cells that were not contacted with gibberellin, and
   (d) isolating the genes identified in step (c);
[3] a method for determining the presence or absence of gibberellin responsiveness in a test gene, wherein the method comprises the steps of:
   (a) contacting cultured plant cells with gibberellin, and
   (b) measuring the expression of a test gene in the cultured plant cells contacted with gibberellin,
      wherein the test gene is judged to be gibberellin responsive when its expression is different from that in cells not contacted with gibberellin; and
[4] the method according to any one of [1] to [3], wherein plant cells cultured in a medium without auxins are used.

The present invention provides methods for identifying gibberellin responsive genes using cultured plant cells. In the methods for identification of this invention, the cultured plant cells are first contacted with gibberellin.

In this invention, “gibberellin responsive genes” means genes that show gibberellin-dependent changes in expression. Herein, “changes in expression” means an increase or decrease in expression. An increase or decrease in expression includes a transient increase or decrease. Decrease of expression includes complete blocking of expression.

In this invention, the term “gibberellins” refers to four-membered ring diterpenoid carboxylic acids comprising a basic ent-gibberellane structure. To date, about 120 different chemical species of gibberellin are known, including GA1, GA3, GA4, GA13, GA17, GA18, GA19, GA20, GA21, and GA22. In this invention, gibberellins include those described above; however, they are not limited thereto.

When the gibberellins of this invention are natural type gibberellins, they can be obtained by purification from desired plants, according to known methods in the art. In the cases of GA1, GA3, and GA4, products commercially available from Wako Pure Chemicals can be used (catalogue Nos. 077-04191, 075-02811, and 072-02821). In addition, methods for preparing synthetic gibberellins are well known to those skilled in the art.

The plant species from which the cultured cells used in this invention are derived are not particularly limited. Examples of plants include useful agricultural crop plants such as cereals, vegetables and fruit trees, and horticultural plants such as ornamental foliage plants. More specifically, rice, corn, wheat, barley, rapeseed, soybean, cotton, tomato, potato, chrysanthemum, rose, carnation, and cyclamen can be employed as examples. Preferably, the plant is a gramineous plant, and most preferably one being rice.

The methods for preparing the cultured plant cells of this invention are not particularly limited. For example, cultured cells of rice (mostly cv. Nipponbare) can be obtained by incubating dehulled seeds (husked rice) on agar medium (1% agar) containing 2 mg/L of 2,4-D (2,4-dichlorophenoxyacetic acid) in commercially available MS medium, kept in the dark at 25° C. for about two weeks to form calluses, followed by successively culturing these calluses in liquid L medium (similar to N6 medium) containing 2,4-D (1 mg/L). Cells are transferred to new medium once a week and containers are rotated at 120 rpm at 25° C. The above-mentioned L medium contains 2830 mg/L of KNO₃, 463 mg/L of (NH₄)₂SO₄, 166 mg/L of CaCl₂.2H₂O, 185 mg/L of MgSO₄.7H₂O, 400 mg/L of KH₂PO₄, 5.6 mg/L of FeSO₄.7H₂O, 7.5 mg/L of Na₂EDTA, 4.4 mg/L of MnSO₄.4H₂O, 1.5 mg/L of ZnSO₄.7H₂O, 0.8 mg/L of KI, 1.6 mg/L of H₃BO₃, 0.025 mg/L of CuSO₄.5H₂O, 0.25 mg/L of NaMoO₄.2H₂O, 0.025 mg/L of CoCl₂.6H₂O, 0.5 mg/L of nicotinic acid, 0.1 mg/L of thiamine-HCl, 0.5. mg/L of pyridoxine-HCl, 100 mg/L of myo-inositol, 2 mg/L of glycine, 1 mg/L of 2,4-D, and 30 g/L of sucrose. L medium can be prepared by using KOH to adjust the pH of this mixture to 5.8, and then autoclaving.

Furthermore, the culture conditions of the cultured plant cells are not particularly limited. Culture conditions suited to the desired plant cells can be selected as appropriate.

Prior to being used to detect gene expression, plant cells are pre-cultured in a medium that does not contain substances that inhibit response to gibberellin. Preferably, the medium does not contain auxins. In this invention, the term “auxin” refers to any one of the plant hormones that are synthesized in the tips of plant stems and that stimulate/regulate plant growth with their polar movement, including synthetic substances that show equivalent activities. In this invention, auxins are not particularly limited, and natural auxins such as indoleacetic acid, as well as synthetic auxins such as 2,4-dichlorophenoxyacetic acid and naphthaleneacetic acid can be employed as examples.

Contacting gibberellin with cultured plant cells is usually carried out by adding gibberellin to a medium for cultured plant cells, but the method of contact is not particularly limited as long as it can induce response to gibberellin in cultured plant cells.

In the method for identification of this invention, gene expression is detected in cultured plant cells that have been contacted with gibberellin.

“Gene expression” as used in this invention includes both transcription and translation. Methods for detection that use the amount of transcription product (mRNA amount) as an indicator include the DNA microarray method and the RNA display method; however, the methods are not limited thereto. In the DNA microarray method, gene expression can be detected by first preparing a single-stranded cRNA probe by reverse transcribing poly(A)-RNA extracted from cells, in the presence of cy5-dCTP, and then analyzing the results of hybridization using an array scanner (Microarray Scanner FLA8000 (Fujifilm)). In the RNA display method, gene expression can be detected by using PCR with an appropriate primer to amplify reverse-transcripts of the extracted RNA, and then comparing the products.

Methods for detection using the amount of translation product as an indicator are not particularly limited. For example, protein-containing fractions can be collected according to standard methods well known in the art and the expression of these proteins can be detected using electrophoretic methods such as SDS-PAGE and two-dimensional electrophoresis.

In the methods for identification of this invention, genes that show a change in expression compared to when there is no contact with gibberellin (a control) are identified. When the expression of a certain gene is increased compared to that of a control, the gene is identified as a gene whose expression is induced in response to gibberellin. On the other hand, when the expression of a certain gene is decreased compared to that of a control, the gene is identified as a gene whose expression is suppressed in response to gibberellin.

Furthermore, this invention provides methods of screening for gibberellin responsive genes. These screening methods isolate the genes identified by the above-described methods. Methods for isolating genes are not particularly limited. For example, when the DNA microarray method is used to detect gene expression, the desired gene can be isolated by obtaining a clone (e.g., from a gene bank) that corresponds to spots judged to be induced. When the RNA display method is used, bands judged to be amplified are cut from the gel, amplified by PCR, and then cloned. Full-length cDNA chains can then be obtained by screening a library. Induction of gibberellin-dependent expression of an isolated gene is preferably confirmed using methods such as Northern hybridization.

The present invention also provides methods for determining the presence or absence of gibberellin responsiveness in test genes.

In the methods for determination of this invention, cultured plant cells are first contacted with gibberellin. The test genes are not particularly limited. Any gene for which evaluation of the presence or absence of gibberellin responsiveness is desired can be used as a test gene. The source and preparation of cultured plant cells, chemical species of gibberellin, and the contact of gibberellin with cultured plant cells are similar to those described above in the identification methods of this invention.

In the methods for determination of this invention, expression of test genes is then measured in the cultured plant cells contacted with gibberellin.

Expression of a test gene can be detected at either the transcriptional or translational level, as in the identification methods of the present invention. Detection at the transcriptional level can be carried out as follows: Standard methods can be used to extract mRNA from cultured cells contacted with gibberellin, Northern hybridization can then be carried out on this mRNA, using as the probe a polynucleotide that hybridizes to the mRNA corresponding to the test gene. RT-PCR method can also be applied, wherein the mRNA extracted as described above is used as a template, and a pair of oligonucleotides that hybridize to the mRNA corresponding to the test gene is used as the primers to carry out the polymerase chain reaction.

Detection at the translational level can be carried out by western blotting using an antibody against a protein encoded by the test gene, and by analyzing the positions of spots in two-dimensional electrophoresis, etc. Antibodies for detection can be prepared by a method known to those skilled in the art.

In the methods for determination of this invention, when the expression of a test gene is changed compared to when there is no gibberellin contact (a control), the gene is judged to possess gibberellin responsiveness. In other words, when the expression of a test gene is increased compared to that of a control, the gene is identified as a gene whose expression is induced in response to gibberellin. On the other hand, when expression of a test gene is decreased compared to that of a control, the gene is identified as a gene whose expression is suppressed in response to gibberellin.

### BRIEF DESCRIPTION OF THE DRAWINGS

FIG. 1 depicts a comparison of the amino acid sequences encoded by the CIGR1 gene and CIGR2 gene with those of the GRAS family. SLR1 (OsGAI) indicates the rice gibberellin signal repressor (SEQ ID NO: 5) and Tomato Ls indicates the tomato lateral bud suppressor (SEQ ID NO: 6). Amino acid residues conserved in all four sequences are indicated by *, and amino acid residues conserved in three sequences are indicated by *.

FIG. 2 is the continuation of FIG. 1.

FIG. 3 depicts photographs showing the results of genomic Southern hybridization of the CIGR1 gene and CIGR2 gene. A: CIGR1 gene; B: CIGR2 gene.

FIG. 4 shows the phylogenetic relationship of the CIGR1 gene and CIGR2 gene at the amino acid level. AtSCR: Arabidopsis SCARECROW; AtSCLn: SCARECROW-like genes; AtGRS: an AtGAI-like gene of unknown function; AtGRA: an Arabidopsis gibberellin signal repressor; AtGAI: an Arabidopsis gibberellin signal repressor (functional share with GRA is unknown); OsSLR: rice gibberellin signal repressor; Tomato Ls: tomato lateral bud suppressor; AtPAT: Arabidopsis light signal transduction factor; AtSCL21: an Arabidopsis SCARECROW-like gene (function unknown); CIGR2: a rice gene reported in this study; AtSCL13: an Arabidopsis SCARECROW-like gene (function unknown); AtSCL5: an Arabidopsis SCARECROW-like gene (function unknown); CIGR1: a rice gene reported in this study.

FIG. 5 depicts photographs representing the nuclear localization of the CIGR1 gene and CIGR2 gene. A fused gene 35S/CIGR1/GFP or 35S/CIGR2/GFP was introduced into onion epidermal cells using particle gun method. A laser confocal microscope was used for observations. The fused gene 35S/GFP was used as a control. a: 35S/GFP; b: 35S/CIGR1/GFP; c: 35S/CIGR2/GFP.

FIG. 6 depicts photographs representing CIGR1 gene and CIGR2 gene responsiveness to chitin oligomers. a: time-courses (in minutes) of expression induced by chitin heptamer treatment. b: induction activities of chitin oligomers and chitosan oligomers.

FIG. 7 depicts photographs representing the effect of 2,4-D on the responses of the CIGR1 gene and CIGR2 gene to GA3. The times indicated are times after GA3 treatment.

FIG. 8 depicts photographs and a diagram representing the effect of GA3 concentration on the induction of expression of the CIGR1 gene and CIGR2 gene. a: photographs representing the results of Northern blot hybridization analysis of total RNA extracted after ten minutes of GA3 treatment at various concentrations (expressed in M). b: diagram representing the results of quantitative determination of signals in ‘a’ using an image analyzer. Squares indicate the CIGR1 gene. Open triangles indicate the CIGR2 gene.

FIG. 9 depicts diagrams and photographs representing the physiological activities of gibberellins and expression of genes. Total RNA was extracted after treating cultured rice cells with active gibberellin species (GA1, GA3, or GA4) or inactive gibberellin species (GA13 or GA17) for ten minutes. The photographs show the results of analysis by Northern blot hybridization.

FIG. 10 depicts photographs representing the expression of the CIGR1 gene and CIGR2 gene in green leaves of rice after GA3 treatment. After spraying rice plants with GA3, the third and fourth leaves were sampled at certain intervals. Total RNA extracted from the third and fourth leaves was analyzed using Northern blot hybridization.

FIG. 11 depicts photographs representing the effects of protein kinase inhibitors on elicitor response and gibberellin response. The following compounds were added ten minutes before treatment with chitin heptamer (GN7) or gibberellin (GA3). (A): the positive elicitor response control; (B) : okadaic acid (1 μM) ; (C) : lavendustin A (30 μM) ; (D) : K-252a (20 μM).

### BEST MODE FOR CARRYING OUT THE INVENTION

Herein below, the present invention will be specifically described with reference to Examples; however, it is not to be construed as being limited thereto. The Examples were carried out using the materials and methods described below.

(1) Rice cultured cells: The cultured cells of rice were induced from germinating rice seeds (Oryza sativa cv. Nipponbare) on an N6 agar plate containing 1 ppm 2,4-D (2,4-dichlorophenoxyacetic acid). The cells were successively cultured in liquid N6 medium as follows: Once a week, cells of about 1 ml in volume were transferred to 150 ml of N6 medium. Every two weeks, cell clusters were passed through a 20-mesh metal screen to reduce their size. Cells which had been transferred to 30 ml of medium with no screen treatment, and then shake-cultured for four to six days, were used for extraction of RNA for Northern blotting.

(2) RNA extraction: The extraction of total RNA from cultured cells and green leaves of rice was carried out according to the phenol-SDS method. A Polytron homogenizer was used to homogenize living tissue in the presence of about tenfold or more volume to tissue-volume of phenol (about 90% saturated with water), and a volume of buffer solution (50 mM Tris-HCl pH 9.0, 1% SDS, 50 mM NaCl) equal to this phenol volume. The aqueous phase obtained by centrifugation was then repeatedly extracted with phenol. Total nucleic acid was precipitated by adding 0.6 volume of isopropanol to the extract and stirring the mixture. After dissolving the precipitate in water, a 0.25 volume of 10M LiCl was added, and the mixture was cooled on ice. High molecular weight RNA was collected by centrifugation, washed with 70% ethanol, and then dissolved in a small volume of water to be used as an RNA sample.

(3) A method for gibberellin treatment of cultured cells and green leaves: The cultured cells were pre-washed several times with 2,4-D-free N6 medium, and then suspended in the same medium and shaken for two hours at 25° C. Gibberellin (GA3 dissolved in ethanol) was added to make up each final concentration, cells were cultured with shaking for predetermined periods, and then used for RNA extraction. Nipponbare plants grown for three weeks after seeding were sprayed with gibberellin. The third and fourth leaves were collected at certain intervals, and total RNA was extracted. Elicitor treatment was carried out by directly adding aqueous elicitor solution to cultured cells of rice.

(4) Hybridization: RNA for Northern hybridization was denatured using the glyoxal method. 2.7 μl of glyoxal (final concentration, 1M), 1.6 μl of sodium phosphate (pH 7.0, final concentration, 10 mM), and 8 μl of dimethylsulfoxide (final concentration, 50%) were added to 10 μg of total RNA (3.7 μl). The mixture was kept at 50° C. for one hour and then subjected to electrophoresis in 1.4% agarose (10 mM sodium phosphate, pH 7.0). After electrophoresis, RNA was blotted onto a nylon membrane (Biodyne A). The RNA was fixed onto the membrane by treatment for two hours at 80° C. Hybridization was carried out in a mixture of 50% formamide, 0.1% SDS, 0.1 mg/ml salmon sperm DNA, 5×SSPE (0.9 M NaCl, 50 mM sodium phosphate, 5 mM EDTA, pH 7.4), and 5× Denhardt's solution (0.1% bovine serum albumin, 0.1% Ficoll, 0.1% polyvinylpyrrolidone) at 42° C. for one day. The membrane was then washed with 0.1×SSC (15 mM NaCl, 1.5 mM sodium citrate) three times for five minutes each time at room temperature, and twice for 30 minutes each time at 65° C., and then exposed on x-ray film.

(5) DNA microarray analysis: Microchips (http://cdna01.dna.affrc.go.jp/RMOS/index.html) were prepared using rice ESTs (1265 clones) obtained from the Rice Genome Project. Single-stranded cDNA probes were prepared by reverse transcribing poly(A)-RNA extracted from cells that did not undergo elicitor treatment, and from those that underwent a 15-minute elicitor treatment in the presence of cy5-dCTP. The results were analyzed using Microarray Scanner FLA8000 (Fujifilm).

### EXAMPLE 1

Chitin oligomers (N-acetylchitooligosaccharides) are a major component of the cell wall of rice blast disease fungus, and at low concentrations induce various defense responses in cultured cells of rice. In other words, these substances are known to act as powerful elicitors (substances that induce biological defense responses) (Yamada, A., Shibuya, N., Kodama, O., and Akatsuka, T., “Induction of phytoalexin formation in suspension-cultured rice cells by N-acetylchitooligosaccharides”, Biosci. Biotech. Biochem. (1993) 57, 405-409). In the process of chitin oligomer action, known defense-related enzyme genes, PAL, chitinase, and glucanase, have been shown to be expressed (He, D.-Y., Yazaki, Y., Nishizawa, Y., Takai, R., Yamada, K., Sakano, K., Shibuya, N., and Minami, E., “Gene activation by cytoplasmic acidification in suspension-cultured rice cells in response to the potent elicitor, N-acetylchitoheptaose”, Mol. Plant-Microbe Int. (1998) 12, 1167-1174;Nishizawa, Y. , Kawakami, A., Hibi, T., He, D.-Y., Shibuya, N., and Minami, E., “Regulation of the chitinase gene expression in suspension-cultured rice cells by N-acetylchitooligosaccharides: differences in the signal transduction pathways leading to the activation of elicitor-responsive genes”, Plant Mol. Biol. (1999) 39, 907-914). In addition, using more rapid time-courses, three novel early genes, EL2, EL3, and EL5, were found to be expressed (Minami, E., Kuchitsu, K., He, D.-Y., Kouchi, H., Midoh, N., Ohtsuki, Y., and Shibuya, N., “Two novel genes rapidly and transiently activated in suspension-cultured rice cells by treatment with N-acetylchitoheptaose, a biotic elicitor for phytoalexin production”, Plant Cell Physiol. (1996) 37, 563-567; Takai, R., Hasegawa, K., Kaku, K., Shibuya, N., and Minami, E. , “Isolation and analysis of expression mechanisms of a rice gene, EL5, which shows structural similarity to ATL family from Arabidopsis, in response to N-acetylchitooligosaccharide elicitor”, Plant Sci. (2001) 160, 577-583).

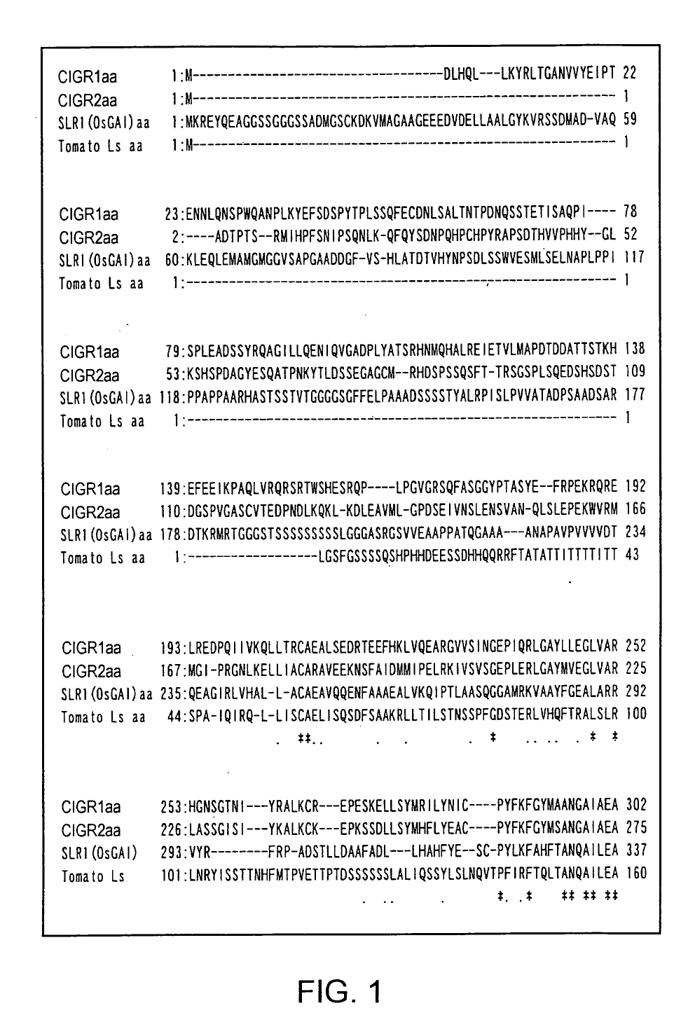

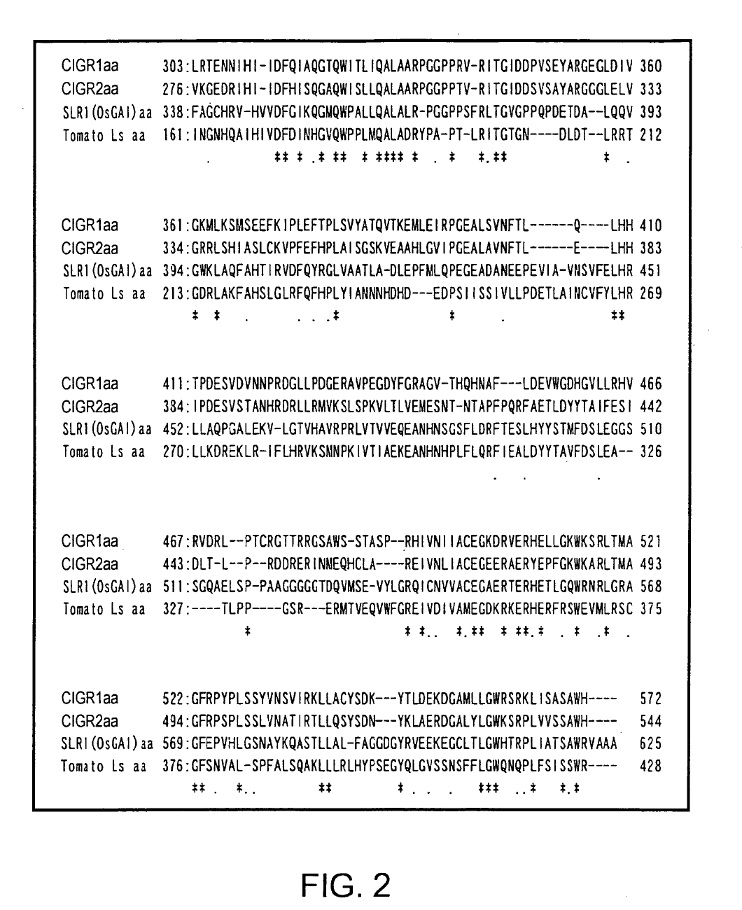

From the MAFF Genebank, the present inventors obtained two ESTs (c72495 and AU094860) whose DNA microarray signals were significantly increased after 15-minutes of elicitor treatment, and determined their entire nucleotide sequences. These deduced amino acid sequences were searched for homology with known sequences. The search results indicated that the two sequences comprised homology with known GRAS family genes (FIG. 1 and FIG. 2).

The GRAS family takes its name from the capital letters of its members; a gene which regulates the formation of radial structures of Arabidopsis roots and stems and is assumed to be a transcription factor (SCARECROW), and two genes that down regulate gibberellin signals in Arabidopsis (GAI and RGA) (Di Laurenzio, L. , Wysocka-Diller, J., Malamy, J. E., Pysh, L., Helariutta, Y., Freshour, G., Hahn, M. G., Feldmann, K. A. and Benfey, P. N., “The SCARECROW gene regulated an asymmetric cell division that is essential for generating the radial organization of the Arabidopsis root”, Cell (1996) 86, 423-433; Peng, J., Carol, P., Richards, D. E., King, K. E., Cowling, R. J., Murphy, G. P., and Harberd, N. P., Genes and Development, (1997) 11, 3194-3205; Silverstone, A. L., Ciampaglio, C. N., and Sun, T.-P., “The Arabidopsis RGA gene encodes a transcriptional regulator repressing the gibberellin signal transduction pathway”, The Plant Cell (1998) 10, 155-169; Pysh, L. D., Wysocka-Diller, J. W., Camilleri, C., Bouchez, D., and Benfey, P. N., “The GRAS gene family in Arabidopsis: sequence characterization and basic expression analysis of the SCARECROW-LIKE genes”, The Plant J. (1999) 18, 111-119). The amino acid sequences of the GRAS family are characterized by N-terminal regions with low homology, a leucine heptad structure, VHIID domain, etc. These structural characteristics are not known in animals and microorganisms. They are unique to plants.

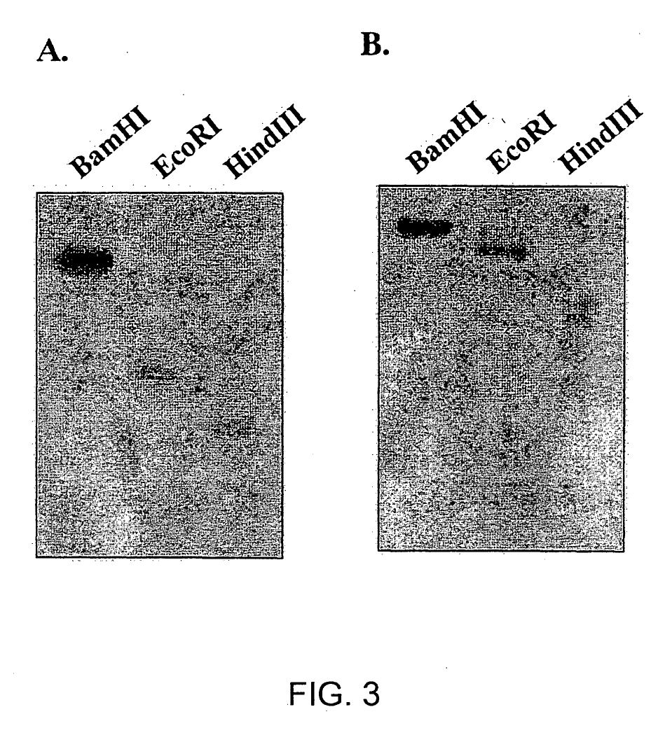

The two genes induced by elicitor treatment comprised VHIID domains and C-terminal amino acid sequences that are highly conserved among SCARECROW and its family. However, they did not comprise the typical leucine heptad structure. Also absent was the DELLA sequence, which is characteristic of gibberellin signal repressors and plays important roles in catching the gibberellin signal (Harberd, N. P., King, K. E., Carol, P., Cowling, R. J., Peng, J., and Richards, D. E., BioEssays (1998) 20, 1001-1008). Hereafter, C72495 is referred to as the CIGR1 gene (its nucleotide sequence and amino acid sequence are shown in SEQ ID NOs: 1 and 2, respectively), and AU94860 is referred to as the CIGR2 gene (its nucleotide sequence and amino acid sequence are shown in SEQ ID NOs: 3 and 4, respectively). These two genes are 57% homologous at the nucleotide level and 40% homologous at the amino acid level. The results of genomic Southern hybridization suggest that a single copy of each of these genes is present (FIG. 3).

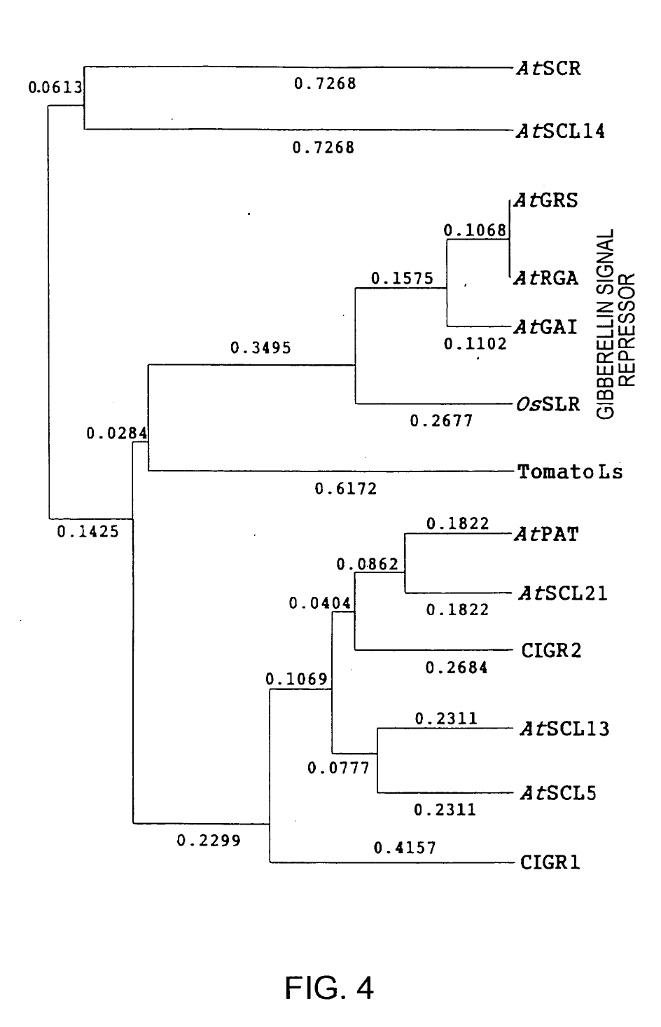

FIG. 4 depicts the estimated molecular phylogenetic tree of the CIGR1 and CIGR2 gene products and those of the GRAS family gene products with known structures. CIGR2 is thought to be somewhat closer to Arabidopsis AtSCL5 than to CIGR1. It is also suggested that gibberellin signal repressors form a single subfamily, and that rice OsGAI (SLR) is closer to the corn repressor and Arabidopsis repressor than to the CIGR1 gene and CIGR2 gene.

### EXAMPLE 2

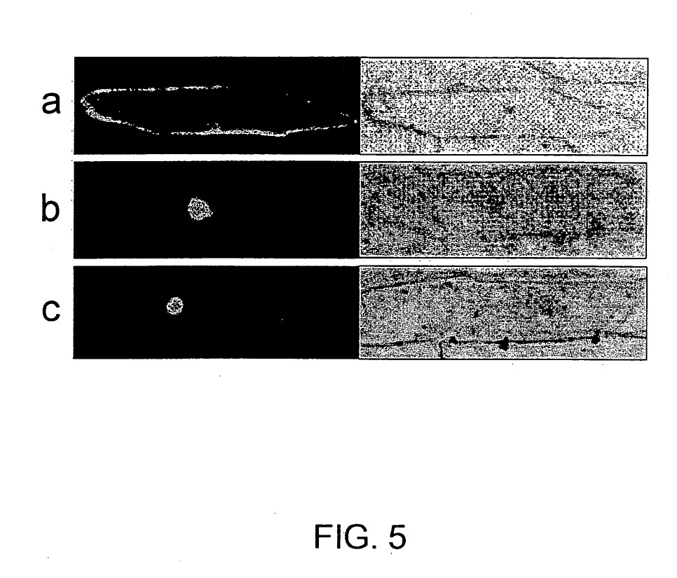

The gene products of the GRAS family are regarded as transcriptional regulators, but their involvement in the regulation of gene expression is unknown. Arabidopsis SCARECROW does not comprise the typical nuclear localization signal. However, it is estimated to be a transcription factor because its N-terminal region is rich in serine, threonine, proline, and glutamine. The GAI gene, which is an Arabidopsis gibberellin signal repressor, and the GRS gene, which is very similar to that, comprise sequences like nuclear localization signals (Peng, J., Carol, P., Richards, D. E., King, K. E., Cowling, R. J., Murphy, G. P., and Harberd, N. P., Genes and Development (1997) 11, 3194-3205). Furthermore, experiments using a GFP-fusion protein showed that RGA, another repressor, was localized in the nucleus (Silverstone, A. L., Ciampaglio, C. N., and Sun, T-P., “The Arabidopsis RGA gene encodes a transcriptional regulator repressing the gibberellin signal transduction pathway”, The Plant Cell (1998) 10, 155-169). To analyze the intracellular localization of the CIGR1 and CIGR2 genes, the particle gun method was used to introduce chimeric plasmids whose translation regions were connected in-frame to GFP. The behavior of the fusion proteins was monitored using GFP fluorescence, and an image of nuclear localization of a CIGR2-GFP fusion protein was observed (FIG. 5). Since this kind of image was not observed when only GFP was introduced, it was concluded that the product of CIGR2 gene was localized in the nucleus.

### EXAMPLE 3

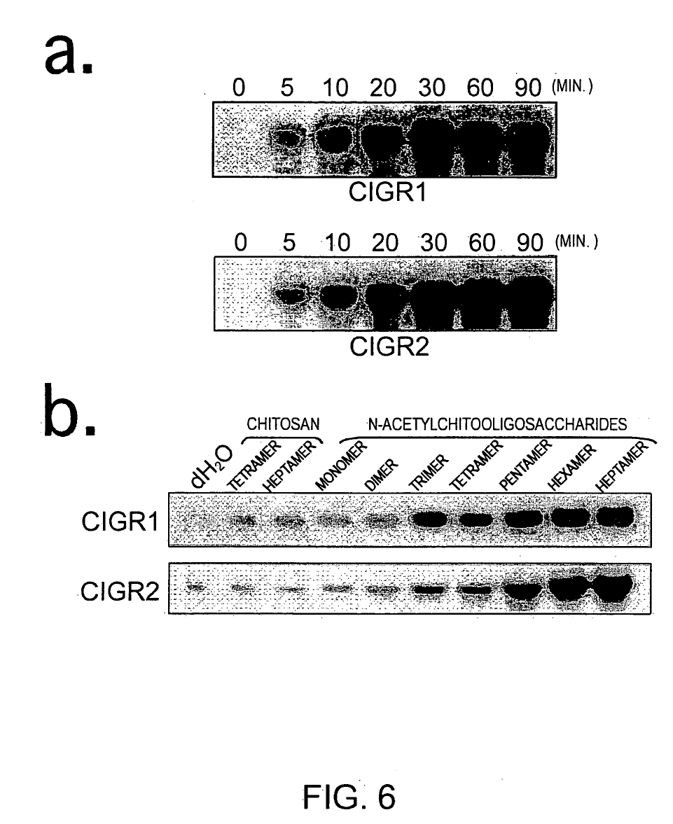

Both the CIGR1 gene and CIGR2 gene were identified as elicitor responsive genes using DNA microarray analysis. The elicitor responsiveness of these two genes was analyzed using Northern blot hybridization. Significant increases in mRNA amount were observed in both genes five minutes after treatment with chitin heptamers. Expression continued to increase for up to 90 minutes (FIG. 6a). It has been reported that the elicitor activity of chitin oligomers in rice depends on their size; that is, chitin heptamers and octamers comprise the strongest effects, and chitosan oligomers that are deacetylated derivatives comprise very low activities. Therefore, the present inventors studied the induction of expression of these two genes when cells were treated with chitin oligomers from monomers to heptamers, and tetramer and heptamer chitosan oligomers. Both genes showed the strongest responses to chitin heptamers. They showed no significant response to chitosan oligomers (FIG. 6b).

### EXAMPLE 4

Ashikari et al. (Ashikari, M., Wu, J., Yano, M., Sasaki, T., and Yoshimura, A., “Rice gibberellin-insensitive dwarf mutant gene Dwarf 1 encodes the alpha-subunit of GTP-binding protein”, Proc. Natl. Acad. Sci. U.S.A. (1999) 96, 10284-10289) and Fujisawa et al. (Fujisawa, Y., Kato, T., Ishikawa, A., Kitano, H., Sasaki, T., Asahi, T., and Iwasaki, Y., “Suppression of the heterodimeric G protein causes abnormal morphology, including dwarfism, in rice”, Proc. Natl. Acad. Sci. U.S.A. (1999) 96, 7575-7580) both independently discovered that gene D1, which causes dwarf mutant d1 in rice, encodes the α-subunit of the trimeric G protein. In animal cells, trimeric G proteins are known to couple with seven-transmembrane receptors and to play important roles in the transduction of extracellular information (Neer, E. J., “Heterotrimeric G proteins: organizers of transmembrane signals”, Cell (1995) 50, 1011-1019).

Analysis using inhibitors and activators suggested that the G proteins were involved in the transduction of elicitor signals (Legendre, L., Heinsyein, P. F., and Low, P. S., “Evidence for participation of GTP-binding proteins in elicitation of the rapid oxidative burst in cultured soybean cells”, J. Biol. Chem. (1992) 267, 20140-20147). However, some problems were pointed out, such as the specificity of these inhibitors was not always precise (Ephritikhine, G., Pradier, J.-M., and Guern, J., “Complexity of GTP γS binding to tobacco plasma membranes”, Plant Physiol. Biochem. (1993) 31, 573-584). Thus, no clear conclusion was obtained concerning the involvement of the D1 gene product.

Tsukada et al. compared in detail the various elicitor responses of calluses derived from d1 strain seeds with those of the wild type, demonstrating that there was no significant difference between them (Tsukada, K., Ishizaka, M., Fujisawa, Y., Iwasaki, Y., Yamaguchi, T., Minami, E., and Shibuya, N., “Rice receptor for chitin oligosaccharide elicitor does not couple to heterotrimeric G-protein: Elicitor responses of suspension cultured rice cells from Daikoku dwarf (d1) mutants lacking a functional G-protein α-subunit”, Physiol. Plantrum (2002) 116, 373-382).

Preliminary experiments demonstrated that the time-courses of CIGR1 gene and CIGR2 gene elicitor induction in the d1 strain were similar to those in the wild type.

On the other hand, Ueguchi-Tanaka et al. showed that the D1 gene is involved in the transduction of gibberellin signals, using the induction of α-amylase in aleurone in germinating seeds as an indicator (Ueguchi-Tanaka, M., Fujisawa, Y., Kobayashi, M., Ashikari, M., Iwasaki, Y., Kitano, H., and Matsuoka, M., “Rice dwarf mutant d1, which is defective in the α subunit of the heterotrimeric G protein, affects gibberellin signal transduction”, Proc. Natl. Acad. Sci. U. S. A. (2000) 97, 11638-11643). Prior to this study, Schumacher et al. showed that the tomato lateral bud suppressor gene product belonged to the same gene family as SCARECROW (Schumacher, K., Schmitt, T., Rossberg, M., Schmitz, G., and Theres, K., “The Lateral suppressor (Ls) gene of tomato encodes a new member of the VHIID protein family”, Proc. Natl. Acad. Sci. U.S.A. (1999) 96, 290-295). They pointed out the possibility of an interaction between this gene product and gibberellin in the discussion on its role in morphogenesis.

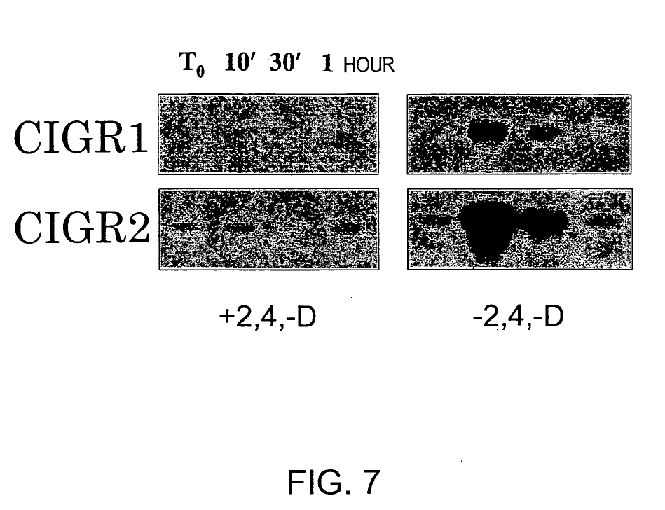

Thus, the present inventors examined the gibberellin responsiveness of the CIGR1 and CIGR2 genes. Suspension-cultured cells were treated with GA3, an active-type gibberellin, and changes in the expression of these two genes were analyzed over time. Expression of both genes was observed for three hours after treatment, and no significant change in expression amount was found. To maintain the cell's ability to divide, the medium used to culture plant cells comprised 2,4-D, auxins that are not easily metabolized by plants. It was thought that 2,4-D might suppress the action of gibberellin. Therefore, cells were pre-washed with 2,4-D-free medium, and treated with GA3 after two hours of pre-culture in the same medium. Transient expression with a maximum at ten minutes after treatment was observed (FIG. 7).

In the prior art, studies on the transduction of gibberellin signals were carried out mostly by analyzing α-amylase induction in the aleurone tissues of cereal seeds. The above-mentioned result is the first example using cultured cells. Responsiveness to gibberellin was then analyzed in more detail by using the expressions of the CIGR1 gene and CIGR2 gene in cultured cells as indicators.

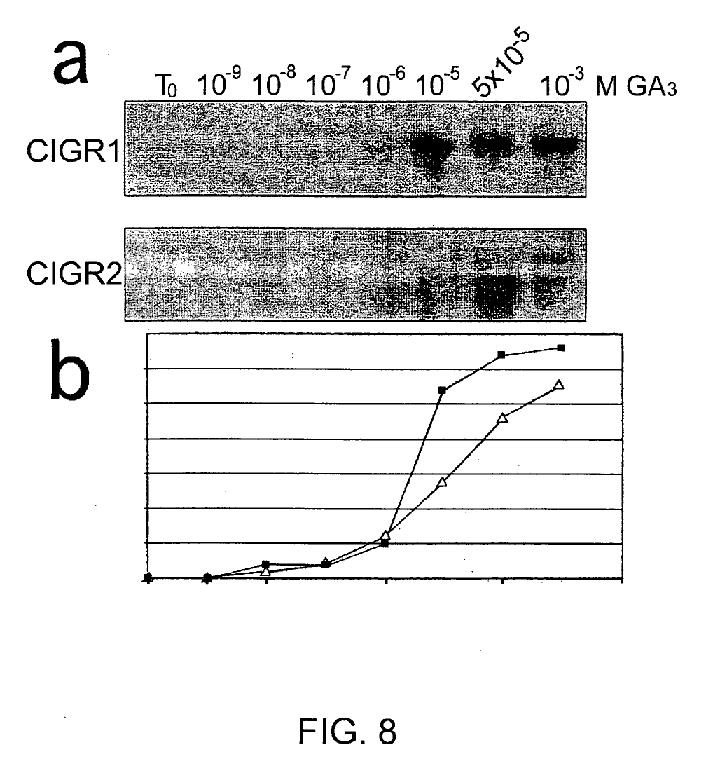

FIG. 8 shows the effect of GA3 concentration on the induction of expression of these two genes. The induction of expression of both genes began to appear with GA3 treatment at 10⁻⁶ M. Induction reached near saturation at 10⁻⁴M. Vishnevetsky et al. reported that CHRC, a carotenoid-associated protein in the chromoplasts of cucumber petals, required at least 10⁻⁷ M GA3 for induction by gibberellin, and that its expression level increased approximately linearly up to 10⁻⁴ M GA3 (Vishnevetsky, M., Ovadis, M., Itzhaki, H., and Vainstein, A., “CHRC, encoding a chromoplast-specific carotenoid-associated protein, is an early gibberellic acid-responsive gene”, J. Biol. Chem. (1997) 272, 24747-24750). Though there were differences in the materials and the genes, they obtained similar results for effective GA3 concentrations.

### EXAMPLE 5

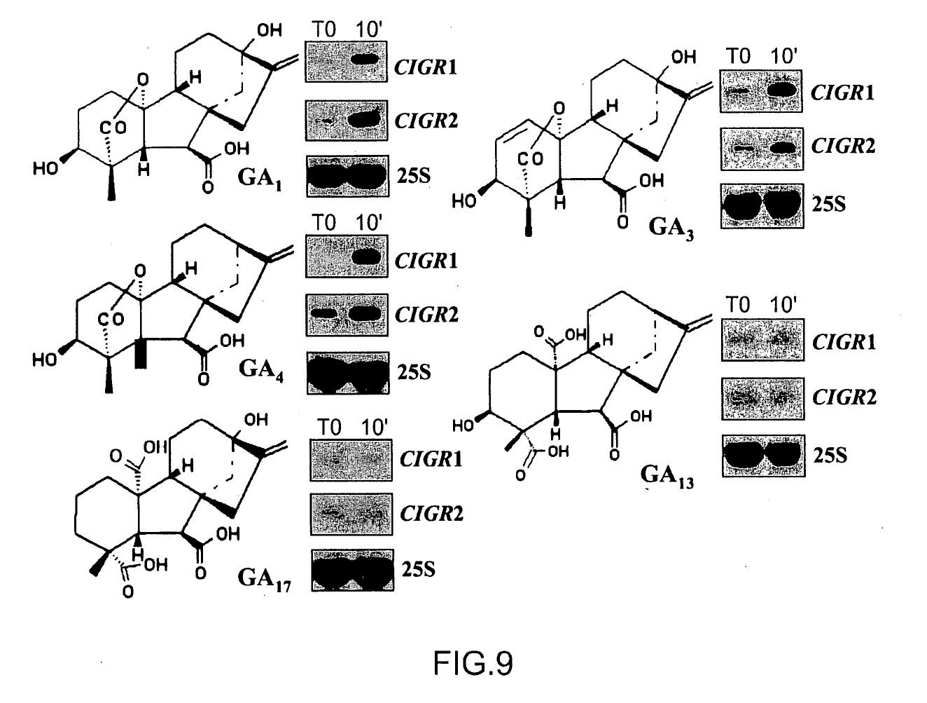

Unlike auxins and cytokinins, gibberellins are compounds defined not by their physiological activities, but by their ent-gibberellane backbone. Therefore, there are large variations in their activities. In order to judge whether induction of CIGR1 gene and CIGR2 gene expression is based on physiological activity or on the ent-gibberellane backbone itself, induction of expression of these genes by active type gibberellins (GA1, GA3, and GA4) and inactive type gibberellins was analyzed (GA13 and GA17) (Crozier, A., Kuo, C. C., Durley, R. C., and Pharis, R. P., “The biological activities of 26 gibberellins in nine plant bioassays”, Canadian J. Botany (1970) 48, 867-877.). The results showed that only active type gibberellins induced the expression of both genes (FIG. 9). This result strongly suggests that the above-mentioned induction of CIGR1 and CIGR2 gene expression by gibberellin occurs via signal transduction mediated by a gibberellin receptor.

### EXAMPLE 6

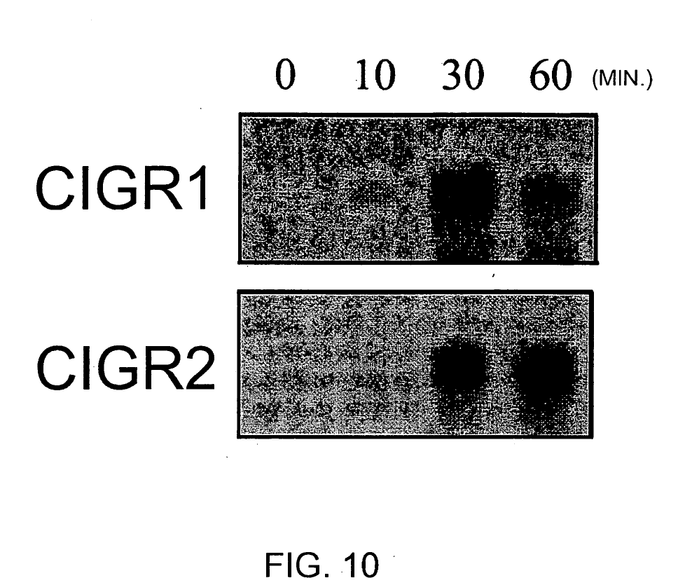

In the presence of auxins, cultured plant cells are thought to be in an undifferentiated state. They also grow under heterotrophic conditions as they are devoid of chloroplasts. Due these and other factors, cultured cells are quite different from whole plants in their histological and physiological characteristics. Therefore, the possibility cannot be denied that the induction of CIGR1 and CIGR2 gene expression by gibberellin is a special phenomenon in cultured cells. Thus, the responses of these two genes to gibberellin were analyzed in the green leaves of rice. The third and fourth leaves of rice, three weeks after seeding, were sprayed with GA3 (50 μM) and total RNA was extracted at certain intervals to analyze changes in the expression level of these genes. Both genes showed very rapid transient expression with a maximum 30 minutes after spraying (FIG. 10). This strongly suggested that the signaling via a gibberellin receptor, found in cultured cells, was not peculiar to cultured cells, and that it also functioned in whole rice plants.

### EXAMPLE 7

Kuo et al. found that the gibberellin's induction of α-amylase in the aleurone layer of wheat was specifically inhibited by okadaic acid, a protein phosphatase inhibitor (Kuo, A., Cappelluti, S., Cervantes-Cervantes, M., Rodriguez, M., and Bush, D. S., “Okadaic acid, a protein phosphatase inhibitor, blocks calcium changes, gene expression and cell death induced by gibberellin in wheat aleurone cells”, The Plant Cell (1996) 8, 259-269.). Okadaic acid is known to inhibit the animal protein phosphatases PP1 and PP2B.

On the other hand, the protein kinase inhibitors staurosporine and K252a hardly inhibited induction. Thus it was presumed that protein phosphorylation, especially by protein phosphatases, was critically involved in signal transduction from gibberellin to the α-amylase gene.

In contrast, our results to date suggest that the induction of expression of elicitor-responsive genes is strongly inhibited by K-252a pretreatment (He, D.-Y., Yazaki, Y., Nishizawa, Y., Takai, R. , Yamada, K., Sakano, K., Shibuya, N., and Minami, E., “Gene activation by cytoplasm acidification in suspension-cultured rice cells in response to the potent elicitor, N-acetylchitoheptaose”, Mol. Plant-Microbe Int. (1998) 12, 1167-1174; Nishizawa, Y., Kawakami, A., Hibi, T., He, D.-Y., Shibuya, N., and Minami, E., “Regulation of the chitinase gene expression in suspension-cultured rice cells by N-acetylchitooligosaccharides: differences in the signal transduction pathways leading to the activation of elicitor-responsive genes”, Plant Mol. Biol. (1999) 39, 907-914).

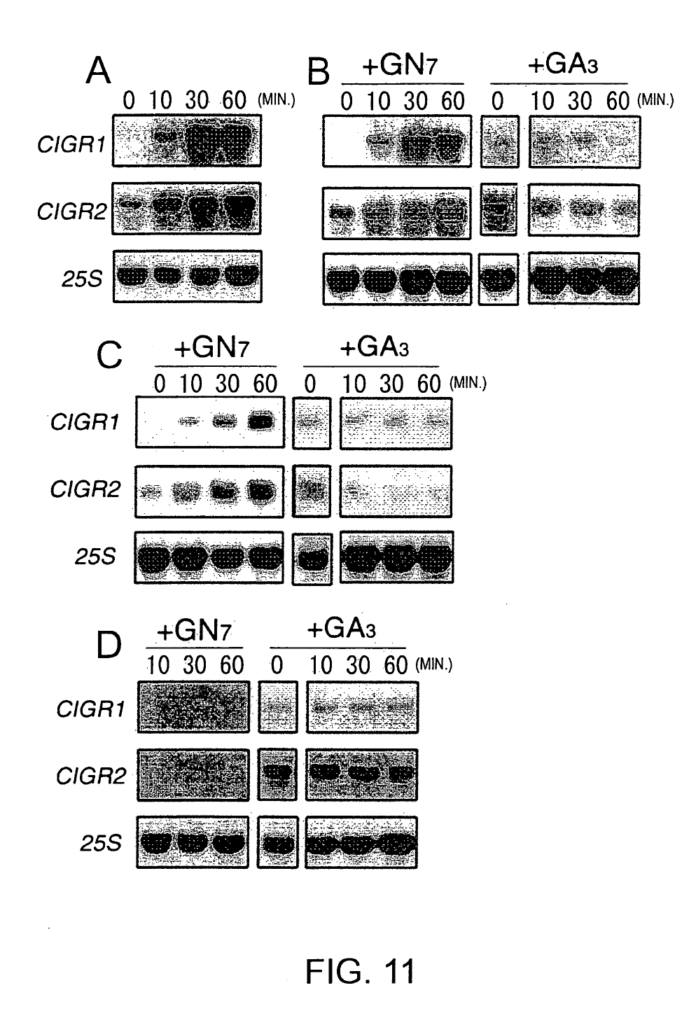

The effects of various inhibitors on gibberellin and elicitor induction of the CIGR1 gene and CIGR2 gene in cultured rice cells were examined. With reference to the results of Kuo et al., cells pretreated with okadaic acid were tested. In these cells, induction of both genes by chitin heptamer was hardly inhibited, however induction by GA3 was inhibited almost completely (FIG. 11B). From these observations, the involvement of protein phosphatase in signal transduction from gibberellin to the CIGR1 and CIGR2 genes in cultured rice cells was presumed. Following these experiments, the effects of protein kinase inhibitors were studied. Lavendustin A, which is known as an inhibitor of receptor-type protein tyrosine kinase, exhibited a pattern of inhibition similar to that of okadaic acid. Lavendustin A hardly inhibited induction by chitin heptamer, but inhibited induction by GA3 almost completely (FIG. 11C). K-252-A, which is thought to inhibit both protein serine/threonine kinase and protein tyrosine kinase, almost completely inhibited the induction of both genes by chitin heptamer and GA3 (FIG. 11D).

α-amylase induction by gibberellin in the aleurone layer of wheat is reported to be inhibited by okadaic acid, but not by K-252-A (Kuo, A., Cappelluti, S., Cervantes-Cervantes, M., Rodriguez, M., and Bush, D. S., “Okadaic acid, a protein phosphatase inhibitor, blocks calcium change, gene expression and cell death induced by gibberellin in wheat aleurone cells”, The Plant Cell (1996) 8, 259-269).

The results shown in FIG. 11 suggest that the induction of the CIGR1 and CIGR2 genes by gibberellin in cultured cells involves not only protein phosphatase, which is inhibited by okadaic acid, but also protein kinase, which is inhibited by K-252a, and lavendustin A. Signal transduction in cultured cells and in the aleurone layer had some aspects in common, and some aspects that were different. Signal transduction from gibberellins is thought to be qualitatively different to that from chitin heptamers.

Richards et al. proposed a hypothesis that the GRAS family corresponds to STATs, which are transcriptional factors found widely in metazoan organisms (Richards, D. E., Peng, J., and Harberd, N. P., BioEssays (2000) 22, 573-577). In STAT C-terminal regions, structures similar to the SH2 region are found, where the SH2 region is a structure common to the STAT family, which are transcription factors in animals. Near the C-terminal of the SH2 region, a tyrosine residue that is highly conserved in animal STATs and susceptible to phosphorylation, and an arginine residue that is supposed to interact electrostatically with the phosphorylated tyrosine, is conserved. Homology in amino acid sequence N-terminal regions is lower than in C-terminal regions. This is generally found in the GRAS family. The homology of the CIGR1 gene and CIGR2 gene at the nucleotide level was 57%. From the result of genomic Southern hybridization, it was estimated that these two genes were present in one copy each (FIG. 3). Five μg of rice genomic DNA was completely digested by restriction enzymes BamHI (B), EcoRI (E), or HindIII (H). The products were separated on agarose gel by electrophoresis, and transferred onto nitrocellulose membranes. Hybridization with ³²P-labelled CIGR1 and CIGR2 resulted in patterns consistent with the cDNA maps.

From these results, each of the full-length cDNA strands was thought to hybridize specifically with the corresponding gene product. Neither of the genes comprises the DELLA sequence, a common structure in the GAI/RGA subfamily, which are negative regulators of gibberellin signals.

Industrial Applicability

The present invention made it possible to comprehensively and conveniently identify and isolate gibberellin responsive genes. In addition, the present invention made it possible to conveniently determine the presence or absence of gibberellin responsiveness in a certain gene. The gibberellin responsive genes identified by the methods of this invention are expected to have various applications, including in the generation of transgenic crops whose germination, elongation, flower bud formation, and so on has been modified.

1. A method for identifying gibberellin responsive genes, wherein the method comprises the steps of:
(a) contacting cultured plant cells with gibberellin,
(b) detecting gene expression in the cultured plant cells contacted with gibberellin, and
(c) identifying genes that have changes in expression when compared with cells that were not contacted with gibberellin.

2. A method of screening for gibberellin responsive genes, wherein the method comprises the steps of:
(a) contacting cultured plant cells with gibberellin,
(b) detecting gene expression in the cultured plant cells contacted with gibberellin,
(c) identifying genes that have changes in expression when compared with cells that were not contacted with gibberellin, and
(d) isolating the genes identified in step (c).

3. A method for determining the presence or absence of gibberellin responsiveness in a test gene, wherein the method comprises the steps of:
(a) contacting cultured plant cells with gibberellin, and
(b) measuring the expression of a test gene in the cultured plant cells contacted with gibberellin, wherein the test gene is judged to be gibberellin responsive when its expression is different from that in cells not contacted with gibberellin.

4. The method according to claim 1, wherein plant cells cultured in a medium without auxins are used.

5. The method, according to claim 2, wherein plant cells cultured in a medium without auxins are used.

6. The method, according to claim 3, wherein plant cells cultured in a medium without auxins are used.


SEQUENCE LISTING (6 sequences)

SEQ ID NO: 1 - DNA, 2135 bp, Oryza sativa L cv Nipponbare
  CDS (137)..(1852)
gcacgagagagaaccagagagagagagagagagagagagagagagagaggagtggaagtggatcggggcatcgctgcggcgtcggggctgtgcagatcggaggaggtcatgaacgactaatctccgttcatttattatggacttgcaccagttattaaagtacagattgactggcgctaacgttgtgtacgaaattcctacagagaacaacttgcaaaactctccctggcaagctaacccactgaagtacgagttcagcgattccccatacacccctctctcttcccaatttgagtgtgacaatttgtctgctcttaccaacactccagataaccagagctctacagaaaccatttcagcccaaccaatctccccattggaagcagacagctcatatagacaggcgggtattcttctccaggagaacattcaagtgggagctgatccgttgtatgctacatcaagacataatatgcaacacgccttacgggagattgagactgttctgatggcacctgatacagatgatgcaacaactagcaccaagcatgagtttgaggaaatcaagcctgctcagcttgtgaggcagcggtcgagaacatggagtcatgaatcacggcagccgttacctggagttggtcggtcacagtttgcatctggtggataccccacagcaagctatgaatttcgtccagagaaacggcaaagggagttaagggaagaccctcagataattgtgaagcagctattaaccaggtgtgctgaggctctgagtgaggacaggacagaggagttccataagcttgttcaggaggctcgtggagtggtctcaatcaacggggaaccaatccaacgtctaggtgcttacctactggagggtttggttgctagacatggaaactctggcacaaacatctaccgtgctctgaagtgccgtgagccagagagcaaggagctcctgtcctacatgagaattctatacaatatctgcccttacttcaagtttggctatatggcagccaatggggcgattgcagaagcattgagaactgagaacaatatccacataattgattttcagattgctcaagggactcaatggatcacactgatacaagcattagctgcaaggcctggtggtcctcctcgtgtgcggatcaccgggatagatgacccagtgtcagagtatgctcgtggtgaaggtcttgacattgtggggaaaatgttgaaaagcatgtctgaagaattcaaaatacctctggagtttacgcctctgtctgtctatgccacacaagtcacgaaagagatgcttgagatcaggccaggtgaagcactgtctgtaaacttcacactccagctacaccacaccccggacgagagcgtggatgtcaacaacccacgcgatggtctgctaccggatggtgaaagggctgtccccgaaggtgactactttggtagagcaggagtcacacaccaacacaacgcctttcttgatgaggtttggggagaccatggagtactactccgccatgttcgagtcgatcgactgccaacctgccgcgggacaacaaggagaggatcagcgtggagcagcactgcctcgccaaggcacatcgtcaacatcatcgcctgcgaggggaaggacagggtggagaggcatgagctgcttggcaagtggaagtcgaggctgaccatggccggcttcaggccttacccgttgagctcgtacgtcaactcggtgataaggaagcttctcgcctgctactccgataaatacacattggatgagaaggacggcgcgatgcttctcggctggaggagcagaaagctgatatctgcttctgcgtggcactgacagtagtatgttgagaagaagaaatgatagagagcatatatatacataccgattccctggtccaatttacagaatgataccctttgatgtaccctcagtttgattgacagcgggtgttgccttcatttatagaccaggatcaaacattgacattagatttgtagagaagttgtatgataataatttctttgcgatttcaatgtatgatgtatccatggctgccatagttaataatattcttgcatagtcaaaaaaaaaaaaaaaaaaaaaaaaaaaaaaa

SEQ ID NO: 2 - protein, 572 aa, Oryza sativa L cv Nipponbare
MDLHQLLKYRLTGANVVYEIPTENNLQNSPWQANPLKYEFSDSPYTPLSSQFECDNLSALTNTPDNQSSTETISAQPISPLEADSSYRQAGILLQENIQVGADPLYATSRHNMQHALREIETVLMAPDTDDATTSTKHEFEEIKPAQLVRQRSRTWSHESRQPLPGVGRSQFASGGYPTASYEFRPEKRQRELREDPQIIVKQLLTRCAEALSEDRTEEFHKLVQEARGVVSINGEPIQRLGAYLLEGLVARHGNSGTNIYRALKCREPESKELLSYMRILYNICPYFKFGYMAANGAIAEALRTENNIHIIDFQIAQGTQWITLIQALAARPGGPPRVRITGIDDPVSEYARGEGLDIVGKMLKSMSEEFKIPLEFTPLSVYATQVTKEMLEIRPGEALSVNFTLQLHHTPDESVDVNNPRDGLLPDGERAVPEGDYFGRAGVTHQHNAFLDEVWGDHGVLLRHVRVDRLPTCRGTTRRGSAWSSTASPRHIVNIIACEGKDRVERHELLGKWKSRLTMAGFRPYPLSSYVNSVIRKLLACYSDKYTLDEKDGAMLLGWRSRKLISASAWH

SEQ ID NO: 3 - DNA, 2204 bp, Oryza sativa L cv Nipponbare
  CDS (301)..(1932)
  misc_feature (2066)..(2066): n is a, c, g or t
cccgcgtccgcccacgcgtccgcaccacctgcttcctccgcggctgcgtgcgccgcctccccaacccttcgtcctccccgccggccgccgctgagatccgacgcgccggagcggagctcgctgagattttgttcctgctggatttctgaaattttactggaggactagagatatcaagtacaccaacaacaaactagtacaaagttttccttccagttaatctgttcctggatctccttgccatacgcgaagcagtggatactatgcagtaattggttaggaggtctttactgaaccataatggctgatactccaacttcccgaatgattcatcccttcagcaatataccgagccaaaacctgaagcagttccagtattctgataatccacagcacccttgccacccttatagggcaccctcagacacccacgttgtgccacatcattatggcctaaagtcacattcaccggatgctggttatgaaagccaggctactcctaacaagtacacattggactcctctgaaggtgctggttgtatgagacatgattctccttccagtcaaagtttcacaaccagaagtggtagccctctatctcaggaagacagccactctgactcgactgatggatctcctgtaggcgcctcctgtgtcactgaggatcctaatgatctgaagcaaaaactgaaggatctcgaggccgttatgcttgggccagactcggaaatagtcaacagccttgagaattcggtggcaaaccaactttccttggagccggagaagtgggtgcgcatgatgggcattcctagaggcaacttgaaagagctactgattgcctgtgctagagctgtggaagagaagaatagctttgctattgatatgatgattccagagctgagaaaaatagtttcggtatctggtgagccacttgagaggttgggagcctacatggtagaagggcttgttgccaggcttgcctcctctggcatttcaatctacaaagctttgaaatgcaaggaaccaaagagctctgaccttctgtcttacatgcacttcctgtatgaggcctgtccctacttcaagtttgggtatatgtcggcaaatggtgctattgcagaggctgtcaagggagaagacaggattcatatcatcgacttccatatctctcaaggggctcagtggatctctctccttcaggcccttgcagccaggcctggtggaccaccaactgtaaggatcactggtattgatgactcagtgtcagcttacgcacgaggcggcgggctagagctggtcggaaggaggctgtcgcacatcgccagcctgtgcaaggtgccctttgaattccacccacttgctatctccggcagcaaggtggaggcagcacatctcggagtcatccccggggaagccctcgccgtgaacttcaccctggagctgcaccacattccagacgagtcggtgagcacggcgaaccaccgtgaccgcctcctaaggatggtgaagagcctgtcgccgaaggtactcaccctggtggagatggagtccaacacgaacacggcgccattcccgcagaggttcgcggagacgctggactactacaccgccatcttcgagtccatcgacctgacgctgccgagggacgacagggagaggatcaacatggagcagcactgcctcgcgagggagatcgtcaacctcatcgcgtgcgagggggaggagagggcggagcggtacgagcccttcggcaagtggaaggcgcgcctcaccatggccgggttcaggccgtcgccgctcagctcgctggtgaacgccaccatcaggacgctgctgcagagctactcggacaactacaagctcgccgagagggatggggcgctctacctcgggtggaagagcaggcccctggtggtctcgtcggcgtggcactagcgttgtgactttgtgaggtgatctgacgacgaacttcccttgtaaccatcttggctgggggctttagaaagctatagtgatcatggactggtaagggtagctgtaacaatggtgttgtaatggtaaggtancggctagggtcaaactcgaaaatgggcaaaatgctgatnaggtgtanaagtgtagtacatgtttnanagtatatgtttcagtttttcttttaaaaaaaaaaaaaaaaaaaaaaaaaaaaaaaaaaaaaaaaaaaaaaa

SEQ ID NO: 4 - protein, 544 aa, Oryza sativa cv Nipponbare
MADTPTSRMIHPFSNIPSQNLKQFQYSDNPQHPCHPYRAPSDTHVVPHHYGLKSHSPDAGYESQATPNKYTLDSSEGAGCMRHDSPSSQSFTTRSGSPLSQEDSHSDSTDGSPVGASCVTEDPNDLKQKLKDLEAVMLGPDSEIVNSLENSVANQLSLEPEKWVRMMGIPRGNLKELLIACARAVEEKNSFAIDMMIPELRKIVSVSGEPLERLGAYMVEGLVARLASSGISIYKALKCKEPKSSDLLSYMHFLYEACPYFKFGYMSANGAIAEAVKGEDRIHIIDFHISQGAQWISLLQALAARPGGPPTVRITGIDDSVSAYARGGGLELVGRRLSHIASLCKVPFEFHPLAISGSKVEAAHLGVIPGEALAVNFTLELHHIPDESVSTANHRDRLLRMVKSLSPKVLTLVEMESNTNTAPFPQRFAETLDYYTAIFESIDLTLPRDDRERINMEQHCLAREIVNLIACEGEERAERYEPFGKWKARLTMAGFRPSPLSSLVNATIRTLLQSYSDNYKLAERDGALYLGWKSRPLVVSSAWH

SEQ ID NO: 5 - protein, 625 aa, Oryza sativa L cv Nipponbare
MKREYQEAGGSSGGGSSADMGSCKDKVMAGAAGEEEDVDELLAALGYKVRSSDMADVAQKLEQLEMAMGMGGVSAPGAADDGFVSHLATDTVHYNPSDLSSWVESMLSELNAPLPPIPPAPPAARHASTSSTVTGGGGSGFFELPAAADSSSSTYALRPISLPVVATADPSAADSARDTKRMRTGGGSTSSSSSSSSSLGGGASRGSVVEAAPPATQGAAAANAPAVPVVVVDTQEAGIRLVHALLACAEAVQQENFAAAEALVKQIPTLAASQGGAMRKVAAYFGEALARRVYRFRPADSTLLDAAFADLLHAHFYESCPYLKFAHFTANQAILEAFAGCHRVHVVDFGIKQGMQWPALLQALALRPGGPPSFRLTGVGPPQPDETDALQQVGWKLAQFAHTIRVDFQYRGLVAATLADLEPFMLQPEGEADANEEPEVIAVNSVFELHRLLAQPGALEKVLGTVHAVRPRIVTVVEQEANHNSGSFLDRFTESLHYYSTMFDSLEGGSSGQAELSPPAAGGGGGTDQVMSEVYLGRQICNVVACEGAERTERHETLGQWRNRLGRAGFEPVHLGSNAYKQASTLLALFAGGDGYRVEEKEGCLTLGWHTRPLIATSAWRVAAA

SEQ ID NO: 6 - protein, 427 aa, Lycopersicon esculentum
LGSFGSSSSQSHPHHDEESSDHHQQRRFTATATTITTTTITTSPAIQIRQLLISCAELISQSDFSAAKRLLTILSTNSSPFGDSTERLVHQFTRALSLRLNRYISSTTNHFMTPVETTPTDSSSSSSLALIQSSYLSLNQVTPFIRFTQLTANQAILEAINGNHQAIHIVDFDINHGVQWPPLMQALADRYPAPTLRITGTGNDLDTLRRTGDRLAKFAHSLGLRFQFHPLYIANNNHDHDEDPSIISSIVLLPDETLAINCVFYLHRLLKDREKLRIFLHRVKSMNPKIVTIAEKEANHNHPLFLQRFIEALDYYTAVFDSLEATLPPGSRERMTVEQVWFGREIVDIVAMEGDKRKERHERFRSWEVMLRSCGFSNVALSPFALSQAKLLLRLHYPSEGYQLGVSSNSFFLGWQNQPLFSISSWR